(19) United States
(12) Patent Application Publication
(10) Pub. No.: US 2022/0273690 A1
(43) Pub. Date: Sep. 1, 2022

(54) Edible Formulations Based on Active Ingredients and Arginine

(71) Applicant: GRAAL S.R.L., Monza (IT)

(72) Inventors: Antonio Enrico Seneci, Monza (IT); Federico Luigi Pialorsi, Milan (IT)

(21) Appl. No.: 17/637,906

(22) PCT Filed: Aug. 27, 2020
(86) PCT No.: PCT/IB2020/058009

§ 371 Date: Feb. 24, 2022

(30) Foreign Application Priority Data
Aug. 29, 2019 (IT) 102019000015192

(51) Int. Cl. A61K 31/7076; A61K 47/18; A61K 9/00; A61K 33/06; A61K 38/06; A61K 31/375; A61K 31/385; A61K 31/205; A61K 31/4045

(52) U.S. Cl. CPC A61K 31/7076; A61K 47/183; A61K 9/0056; A61K 33/06; A61K 38/063; A61K 31/375; A61K 31/385; A61K 31/205; A61K 31/4045

(57) ABSTRACT

The object of the present invention is the use of arginine and/or a salt thereof as a stabilizer of an acid active ingredient having a pKa of between 1 and 5 such as S-Adenosyl-L-methionine, and an edible formulation comprising an acid active ingredient, arginine and/or a pharmaceutically acceptable salt thereof, and at least one physiologically acceptable excipient.

### STATE OF THE ART

The importance of therapeutic adherence represents a constant challenge in the world of pharmaceutical and food industry to improve product compliance, ease of intake, and the “taste” of finished products.

The peculiarity of many acid active ingredients is that they have a very low palatability, especially if administered orally, thus affecting the benefits of a buccal absorption which usually improves the systemic bioavailability performance thereof.

The prior art reports various ways to overcome the acidity of these active ingredients, buffering their acidity thanks to the use of specific additives and/or active substances, which are also able to minimize the unpleasant taste of the active ingredient due to the acidity thereof.

Carbonates or bicarbonates, such as sodium bicarbonate or sodium carbonate, acid amino acids such as L-Arginine or L-Lysine, phosphates or citrates such as tri-sodium phosphate or potassium citrate tribasic and magnesium oxide can be included among the most well-known buffering substances known in the art (EP2370062B1, EP2393475B1).

However, it has been observed that the use of buffering substances, such as lysine or citrates, in oral formulations of active ingredients has the disadvantage of requiring the use of large amounts of such buffering agents, which are not compatible with the obtainment of oral formulations; while the use of magnesium oxide, an extremely poorly soluble excipient, requires particularly long stabilization times, which do not allow its use in granules that should be dissolved in the oral cavity.

Other buffering substances, such as carbonates and bicarbonates, by reacting with the acid active ingredients, generate effervescence, thus limiting the possibility of their use only in effervescent oral formulations, among the various pharmaceutical forms.

It is therefore apparent the need to identify a specific buffering substance that can be used in the preparation of oral formulations comprising high acidity active ingredients, which allows these formulations to be stabilized and which is able to overcome the issues listed above.

### Definitions

Unless otherwise defined, all terms of the art, notations, and other scientific terms used herein are intended to have the meanings commonly understood by those skilled in the art to which this description belongs. In some cases, terms with meanings that are commonly understood are defined herein for clarity and/or ready reference; therefore, the inclusion of such definitions in the present description should not be construed as being representative of a substantial difference with respect to what is generally understood in the art.

The term “simultaneous, separate or sequential use” refers either to the simultaneous administration of the first and second compounds, or in such a way that the two compounds act in the patient's body at the same time, or to the administration of one compound after the other compound in such a way as to provide a therapeutic effect. The terms “comprising”, “having”, “including”, and “containing” are to be construed as open-ended terms (i.e., meaning “comprising, but not limited to”), and are to be considered as a support also for terms such as “consist essentially of”, “consisting essentially of”, “consist of”, or “consisting of”.

The term “physiologically acceptable excipient” refers to a substance devoid of any pharmacological effects of its own, and that does not produce adverse reactions when administered to a mammal, preferably a human being. Physiologically acceptable excipients are well known in the art and are described, for example, in Handbook of Pharmaceutical Excipients, sixth edition 2009, incorporated herein by reference.

The term “pharmaceutically acceptable salts or derivatives” refers to those salts or derivatives having the biological efficacy and properties of the salified or derivatized compound and which do not produce adverse reactions when administered to a mammal, preferably a human being. The pharmaceutically acceptable salts can be inorganic or organic salts; examples of pharmaceutically acceptable salts include, but are not limited to carbonate, hydrochloride, hydrobromide, sulfate, hydrogen sulfate, citrate, maleate, fumarate, trifluoroacetate, 2-naphthalenesulfonate, and para-toluenesulfonate. Further information on pharmaceutically acceptable salts may be found in Handbook of pharmaceutical salts, P. Stahl, C. Wermuth, WILEY-VCH, 127-133, 2008, incorporated herein by reference. Pharmaceutically acceptable derivatives include esters, ethers, and N-oxides.

According to the invention, the term “sublingual” refers to compositions that have to be specifically placed under the tongue in order to dissolve and release the active ingredient.

According to the invention, the term “orosoluble” refers to compositions capable of dissolving and releasing immediately the active ingredient contained therein when contacted with the oral mucosa. In this way the active ingredient can be directly absorbed in the oral mucosa, thus avoiding the hepatic circulation.

According to the invention, the term “orosoluble” is therefore preferably directed to compositions to be inserted into the oral cavity.

According to the present invention, “diluents” mean compounds added when the mass of the active ingredient is not sufficient for preparing the composition.

“Lubricants” mean compounds that prevent powders from adhering to mechanical parts during the production process.

“Aggregating agents” mean compounds that increase the cohesion of powders.

“Disintegrants” mean compounds used to improve the disintegration rate of tablets.

“Filming agents” mean compounds used to film capsules and tablets.

“Coloring agents” mean compounds used to improve the appearance of some pharmaceutical forms, such as tablets, to classify them according to their therapeutic category or to distinguish them from other similar products.

“Sweeteners or flavoring agents” mean compounds added to improve the organoleptic characteristics of the products.

“Antioxidants or antimicrobials” mean compounds used to extend the shelf-life of the product.

According to the invention, the term “high palatability” refers to formulations with a particularly pleasant taste that can be easily administered orally despite the active ingredients contained in them having an unpleasant, bitter and/or acid taste.

### DESCRIPTION

It was surprisingly observed that the addition of arginine to active ingredients having high acidity, or to edible formulations including active ingredients having high acidity, increases solubility and stability of the active ingredient alone or when present in the formulation.

It was also observed that these formulations have an improved palatability.

The possibility to obtain these edible formulations allows to increase the bioavailability of active ingredients which are difficult to administer orally, due to their high acidity and low stability, thus allowing an increased residence time on the oral mucosa thanks to the excellent palatability of the association, and significantly improving therapeutic adherence.

In addition, in an attempt to make formulations based on acid active ingredients acceptable, the person skilled in the art has always had to add excipients such as synthetic sweeteners, flavoring agents, in high amounts.

The market today increasingly requires simple, “clean label” formulations, using the fewest excipients possible, which allow the intake of the active ingredients in a natural and safe way.

An object of the present invention is therefore the use of arginine and/or a pharmaceutically acceptable salt thereof as a stabilizer of an acid active ingredient having a pKa of between 1 and 5, preferably between 1.5 and 4.5.

Preferably said arginine is selected from L-arginine, D-arginine, or DL-arginine, preferably L-arginine.

According to a further preferred aspect of the present invention, said active ingredient is selected from S-Adenosyl-l-methionine or a salt thereof, ascorbic acid, acid amino acids, such as glutamic acid, N-acetyl-carnitine, L-acetyl-cysteine, acid acetyl-salicylic acid, salicylic acid, S-acetyl-1-glutathione, aspartic acid, lipoic acid, hydroxy-methyl-butyrate, carnitine or acid botanical extracts, such as acerola and dog rose extracts.

Even more preferably said active ingredient is S-Adenosyl-l-methionine.

A further object of the present invention is an edible formulation comprising an acid active ingredient with a pKa of between 1 and 5, arginine and/or a pharmaceutically acceptable salt thereof, and at least one physiologically acceptable excipient.

Preferably, said acid active ingredient has a pKa of between 1.5 to 4.5.

In a further preferred aspect, the formulation according to the present invention comprises L-arginine, D-arginine, or DL-arginine, more preferably L-arginine.

The active ingredients that can be used in the edible formulations of the present invention are selected from: S-Adenosyl-l-methionine or a salt thereof, ascorbic acid, acid amino acids, such as glutamic acid, N-acetyl-carnitine, L-acetyl-cysteine, acetyl-salicylic acid, salicylic acid, S-acetyl-1-glutathione, aspartic acid, lipoic acid, hydroxy-methyl-butyrate, carnitine or acid botanical extracts, such as acerola, willow and dog rose extracts.

Preferably said active ingredient is S-Adenosyl-l-methionine.

According to a preferred aspect, S-Adenosyl-l-methionine salts are selected from S-Adenosyl-l-methionine tosylate para-toluenesulfonate, S-Adenosyl-l-methionine butadiene sulfate, S-Adenosyl-l-methionine phytate.

According to a preferred aspect, the edible formulations of the present invention are in the form of orosoluble formulations selected, for example, from orodispersible tablets, sublingual tablets, powders or granulates for sachets and/or stick-packs, or in the form of swallowable formulations selected, for example, from chewable tablets, solutions to drink or syrups.

Preferably, said edible formulations are characterized by having a high palatability, which ensures an easy oral administration.

In the treated subject (human or animal), said high palatability also determines a longer residence time of the composition in the oral cavity, and a consequent lower impulse to swallow, which helps to improve orobuccal absorption rate and bioavailability of the active ingredient.

The aforementioned active ingredients are present in the formulations according to the invention in an amount ranging from 5 to 70% by weight, preferably from 7 to 50% by weight, with respect to the total weight of the formulation.

Arginine or a pharmaceutically acceptable salt thereof are present in the formulation of the present invention in an amount ranging from 5% to 70%, with respect to the total weight of the formulation, preferably from 7 to 45% by weight, even more preferably from 10 to 40% by weight, with respect to the total weight of the formulation.

According to a further preferred aspect, the formulation of the present invention can contain at least one further active ingredient with synergistic action.

Said further active ingredient has a dietary, alimentary, nutraceutical and/or phytotherapeutic activity and are selected from probiotics, mineral salts, tonics, products for cholesterol control, multivitamins and multiminerals, intestinal function adjuvants, vitamins, venotonics, trophic and joint adjuvants, nighttime rest products/tranquilizers, cough products, weight control products, liver function adjuvants, antacids, eye health supplements, anti-hair loss products, immune function adjuvants, products for the urinary tract, memory and cognitive functions adjuvants, products for pregnancy and lactation, multifunctional antioxidants, products for the prostate, products for the throat, anti-flu products, omega 3, products for menopause, peripheral neuropathies adjuvants, products for the treatment of calculosis, such as potassium citrate or other specific supplements.

Preferably, the active ingredients with dietary, alimentary and/or nutraceutical action are selected from vitamins, such as for example Vitamin A, D, E, K, vitamins of group B, pantothenic acid, minerals, such as magnesium, calcium, phosphorus, iron, zinc, copper, manganese, fluorine, selenium, chromium, molybdenum, iodine, boron, potassium, chlorine, sodium, silicon salts, phytoterapics such as botanical extracts of Curcuma longa, Boswellia serrata, Silybum marianum, Ananas comosus, Griffonia simplicifolia, Camelia sinensis, Olea europea and other substances with a nutritional and/or physiological effect, such as essential amino acids, branched amino acids, hydroxy-isocaproic acid (HICA), hyaluronic acid, conjugated linoleic acid (CLA), nervonic acid, alpha-ketoisocaproate (KIC), arabinogalactan, arabinoxylan, arginine alpha-ketoglutarate (AAKG), astaxanthin, beta-alanine, betaine, beta-glucans, butyrate, caffeine, carnosine, chitosan, citicoline, chlorophyll, coenzyme Q10, ubiquinol, choline, collagen, colostrum, chondroitin sulfate, creatine, dimethylglycine, enzymes such as alpha-galactosidase, bromeline, enzymes from fermented maltodextrins, lactase, beta-galactosidase, papain or superoxide dismutase, epigallocathechingallate, phytosterols, flavonoids such as quercetin, quercitrin, rutin, spireoside, hesperidin, hesperitin or diosmin, phospholipids such as phosphatidylcholine, phosphatidylserine or phosphoserine, GABA, gamma oryzanol, glycero-phosphoryl-ethanolamine, glycocyamine, glucomannan, glucosamine, glucuronolactone, glutathione, guar gum, hydroxytyrosol/olive polyphenols, inositol, isoflavones, lactoferrin, lactulose, lycopene, lutein, melatonin, methylsulfonylmethane (MSM), monacolin K from fermented red rice (Monacus purpureus), N-acetyl-D-glucosamine, NADH, naringin, norvaline, nucleotides, fish oil (DHA-EPA), homotaurine, ornithine alpha-ketoglutarate (OKG), palmitoylethanolamide (PEA), PABA, pectin, pycnogenol, policosanol, polydatin, resveratrol, ribose, spermidine, squalene, taurine, theanine, zeaxanthin, or mesozeaxanthin.

In a further preferred aspect of the present invention, said optional active ingredients are present in an amount of between 1 and 50% by weight, preferably between 10 and 40%, with respect to the total weight of the formulation.

Physiologically acceptable excipients that can be used for the edible formulations according to the present invention may be selected from diluents, lubricants, aggregating agents, disintegrating agents, film forming agents, coloring agents, sweeteners or flavoring agents, or antioxidants-antimicrobials.

Preferably, said physiologically acceptable excipients are present in an amount of between 5-80% by weight, preferably between 10-60%, with respect to the total weight of the formulation.

Preferably, the physiologically acceptable excipients that can be used in the formulations of the present invention are selected from sodium, calcium, magnesium, potassium citrate, sodium, calcium, magnesium, potassium phosphate, light magnesium oxide, magnesium hydroxide, magnesium hydroxycarbonate, sodium carbonate, sodium chloride, potassium carbonate, sodium bicarbonate, potassium bicarbonate, adipic acid, citric acid, tartaric acid, alginic acid, stearic acid and salts thereof, oleic acid, 1-leucine, glycerol behenate, hydroxypropylmethylcellulose, hydrogenated vegetable oils, such as palm oil, palm butter, cocoa butter, cocoa paste, cocoa powder, xylitol, maltitol, sorbitol, mannitol, sucralose, acesulfame K, sodium cyclamate, aspartame, sucrose, erythritol, citrus extract, fructose, dextrose, maltose, sprayed malt, sodium aspartate, neoesperidin, maltodextrin, inositol, inulin, brewer's yeast, silica gel, vegetable fibers, such as pea fiber, chitosan, flavoring agents, such as essential oils, powders or the like), peppermint, curly mint or sweet mint, badiana anethole, vanilla, sage, liver, sodium glutamate, fish flour, chicken, grapefruit, peach, limes, or mixtures thereof.

### EXPERIMENTAL SECTION

### Example 1

Some examples of formulations according to the present invention are reported in the following tables.

TABLE 1
Orosoluble granulate in sachet
SAMe- ARGININE

| | Mg |
|---|---|
| SAMe TOSYLATE | 400 |
| L-ARGININE | 300 |
| XYLITOL | 290 |
| FLAVORING AGENT | 5 |
| SILICA GEL | 5 |
| | 1000 |

TABLE 2
Orosoluble granulate in sachet
SAMe- ARGININE

| | Mg |
|---|---|
| SAMe TOSYLATE | 400 |
| L-ARGININE | 300 |
| INOSITOL | 1000 |
| MAGNESIUM | 80 |
| STEARIC ACID | 10 |
| XYLITOL | 393 |
| SUCRALOSE | 2 |
| FLAVORING AGENT | 5 |
| MAGNESIUM STEARATE | 5 |
| SILICA GEL | 5 |
| | 2200 |

TABLE 3
Orosoluble granulate in sachet
SAMe- ARGININE-

| | Mg |
|---|---|
| SAMe TOSYLATE | 400 |
| L-ARGININE | 300 |
| GSH | 250 |
| MAGNESIUM | 80 |
| MANNITOL | 503 |
| ACESULFAME K | 2 |
| FLAVORING AGENT | 5 |
| MAGNESIUM STEARATE | 5 |
| SILICA GEL | 5 |
| | 1550 |

TABLE 4
Orosoluble Tablet
SAMe- ARGININE -

| | Mg |
|---|---|
| SAMe TOSYLATE | 400 |
| DL-ARGININE | 300 |
| CITICOLINE BASE | 500 |
| MAGNESIUM | 80 |
| ISOMALT | 400 |
| ERYTHRITOL | 405 |
| FLAVORING AGENT | 5 |
| MAGNESIUM STEARATE | 5 |
| SILICA GEL | 5 |
| | 2100 |

TABLE 5
Orosoluble granulate in sachet
ACETYLCYSTEINE- ARGININE -

| | Mg |
|---|---|
| ACETYLCYSTEINE | 600 |
| L-ARGININE | 600 |
| VITAMIN C | 80 |
| XYLITOL | 253 |
| MANNITOL | 250 |
| SUCRALOSE | 2 |
| FLAVORING AGENT | 5 |
| MAGNESIUM STEARATE | 5 |
| SILICA GEL | 5 |
| | 1800 |

TABLE 6
Chewable tablets
LIPOIC ACID - ARGININE -

| | Mg |
|---|---|
| LIPOIC ACID | 600 |
| L-ARGININE | 500 |
| L-ACETYL-CARNITINE | 150 |
| ISOMALT | 400 |
| XYLITOL | 395 |
| STEARIC ACID | 40 |
| FLAVORING AGENT | 5 |
| MAGNESIUM STEARATE | 5 |
| SILICA GEL | 5 |
| | 2100 |

TABLE 7
Bottle containing syrup
SAMe- ARGININE-

| | Mg |
|---|---|
| SAMe TOSYLATE | 400 |
| L-ARGININE | 300 |
| VITAMIN C | 60 |
| MELATONIN | 1 |
| FRUCTOSE | 500 |
| SUCRALOSE | 2 |
| SODIUM BENZOATE | 5 |
| FLAVORING AGENT | 5 |
| DISTILLED WATER | 727 |
| | 2000 |

TABLE 8
Swallowable tablet
SAMe- ARGININE

| | Mg |
|---|---|
| SAME TOSYLATE | 400 |
| L-ARGININE | 300 |
| XYLITOL | 290 |
| MAGNESIUM STEARATE | 5 |
| SILICA GEL | 5 |
| | 1000 |

The formulations of Tables 2-8 may optionally contain any one of the optional active ingredients according to the present invention.

### Example 2

TABLE 8
pKa values of the active ingredients used in the compositions

| Active ingredient | pKa | Solubility in water (g/L) |
|---|---|---|
| SAMe | 1.7 | 1.19 |
| Ascorbic Acid | 4.36 | 245 |
| N-Acetyl Cysteine | 3.82 | 5.09 |
| Acetylsalicylic acid | 3.41 | 1.46 |
| S-acetyl-L-glutatione | 1.75 | n.d. |
| Aspartic Acid | 1.7 | 142 |
| Lipoic acid | 4.52 | 0.224 |
| Hydroxy-methyl-butyrate (3- | 4.4 | 15 |
| hydroxy-3-methylbutyric acid) | | |
| Levocarnitine | 4.2 | 5.33 |
| L-Acetyl Carnitine | 4.09 | 142 |
| Glutamic acid | 1.88 | 80.6 |

### Example 3

### Neutralization of an Aqueous Solution of SAM-e Tosylate (Salt)

To demonstrate that not all buffering agents may be used in commercial pharmaceutical forms in order to neutralize the acid pKa of the active ingredients, the following laboratory test was set up to find the exactly measured amount needed to neutralize a solution containing 400 mg of SAMe (salt). After measuring the pH, the solution was gradually added with buffering substance until neutralization and a pH 7 value were reached. As it can be seen from the table below (Table 9), the amount of Lysine and Potassium Citrate tribasic appear to be excessive to consider their use in commercial pharmaceutical forms. More precisely, from the table it can be noted that 15.4 g of Lysine fail to bring the SAMe solution to neutralization (the pH value remains 4).

SAMe tosilate concentration: 400 mg/100 mL

TABLE 9

| | | | Sam-e | | | | | |
|---|---|---|---|---|---|---|---|---|
| | | | tosylate | | | Neutralizing | | |
| | Neutralizing | Neutralizing | weight | Volume | Initial | agents added | Final | Stabilization |
| | Agents | agents Batch | (mg) | (mL) | pH | amount (mg) | pH | time |
| 1 | Arginine | A18120073 | 400 | 100 | 2.08 | 375 | 7.0 | <3 minutes |
| 2 | Lysine | A18080075 | 400 | 100 | 2.08 | 15402 | 4.0 | <3 minutes |
| 3 | Sodium | A18110103 | 400 | 100 | 2.08 | 690 | 7.0 | <3 minutes |
| | bicarbonate | | | | | | | |
| 4 | Sodium | A19010401 | 400 | 100 | 2.10 | 200 | 7.0 | <3 minutes |
| | carbonate | | | | | | | |
| 5 | Sodium | 103\Re2016 | 400 | 100 | 2.09 | 510 | 7.0 | <3 minutes |
| | phosphate | | | | | | | |
| | tribasic | | | | | | | |
| 6 | Potassium | A19050053 | 400 | 100 | 2.10 | 14943 | 7.0 | <3 minutes |
| | citrate tribasic | | | | | | | |
| 7 | Light | A19020294 | 400 | 100 | 2.08 | 44.91 | 7.1 | 60 minutes |
| | magnesium | | | | | | | |
| | oxide | | | | | | | |

Neutralization of an Aqueous Solution Containing Other Acid Principles with L-Arginine.

Based on the excellent neutralization results obtained with L-Arginine, the same method was used to analyze the amount of L-Arginine necessary to neutralize a solution including other acid active ingredients (results below):
An aqueous solution containing 1 g of N-Acetyl-Cysteine is neutralized with 1.086 g of L-Arginine
An aqueous solution containing 1 g of L-Acetyl-Carnitine is neutralized with 0.736 g of L-Arginine

### Example 4

### Taste Test for Palatability of SAMe in the Presence of Different Buffering Agents.

To prove the excellent palatability of the invention, especially when compared with other neutralizing substances, the following experimental test was set up using as the active ingredient SAMe (S-adenosyl-l-methionine tosylate, para-toluenesulfonate), the active with the lowest pKa among those tested and certainly the most critical in terms of flavor and stability.

Single-dose sachets containing 400 mg of SAMe (S-adenosyl-l-methionine tosylate, para-toluenesulfonate) were prepared, in addition to a weighed amount of the various buffers in the table of Example 2, so as to have mixtures at pH 7.

Associations of SAMe with Lysine and Potassium citrate were excluded from the palatability test because the amounts of neutralizing agent required are considered excessive (Lysine does not neutralize the acid solution even at 15.4 g) for pharmaceutical forms for oral intake too.

All the sachets thus obtained were made to taste by 8 people, without them knowing what the neutralizing agent contained in each sachet was.

The volunteers were asked to express an evaluation from 0 to 5, where zero is the lowest score (unpleasant) and 5 the highest score (pleasant), with respect to the following parameters: first feeling, dissolution in the mouth, aftertaste after final dissolution, bitterness, acidity, exothermia, effervescence.

More specifically, scores were given as follow:
First feeling: zero (Unpleasant)-five (Pleasant)
Dissolution in the mouth: Zero (Slightly soluble—Unpleasant)-Five (Very soluble—Pleasant)
Aftertaste after final dissolution: Zero (Unpleasant)-Five (Pleasant)
Bitterness: Zero (Unpleasant—Very bitter)-Five (Pleasant—Slightly bitter)
Acidity: Zero (Unpleasant—Very acid)-Five (Pleasant—Slightly acid)
Exothermia: Zero (Strong exothermic feeling)-Five (Absence of exothermic feeling)
Effervescence: NO (Absence of effervescence)-YES (Presence of effervescence)

The evaluation of the test 13919-4 (SAMe—Sodium Phosphate Tribasic) gave a result of zero (0) due to an intense caustic feeling when in contact with the oral mucosa which did not allow further evaluations.

The average of the scores obtained are reported in the table below.

TABLE 10
Legend of samples used

| TEST ID | ACTIVE | BUFFERING AGENT |
|---|---|---|
| PR-13919-1 | SAMe | L-ARGININE |
| PR-13919-2 | SAMe | SODIUM BICARBONATE |
| PR-13919-3 | SAMe | SODIUM BICARBONATE |
| PR-13919-4 | SAMe | SODIUM PHOSPHATE TRIBASIC |
| PR-13919-5 | SAMe | MAGNESIUM OXIDE |

TABLE 11
Palatable SAMe Taste test for samples described in
Table 10 (average evaluation form 8 volunteers)

| PALATABLE SAMe | | | | | |
|---|---|---|---|---|---|
| EVALUATION FORM | | | | | |
| Taste tests | PR-13919-1 | PR-13919-2 | PR-13919-3 | PR-13919-4 | PR-139191-5 |
| FIRST FEELING | 3.125 | 1 | 1 | 0 | 0.25 |
| DISSOLUTION IN | 4 | 3.25 | 2.5 | 0 | 0.5 |
| THE MOUTH | | | | | |
| AFTERTASTE AFTER | 3.75 | 1.375 | 1 | 0 | 0.625 |
| FINAL DISSOLUTION | | | | | |
| BITTERNESS | 3.125 | 1.5 | 1.25 | 0 | 0.625 |
| ACIDITY | 3.5 | 1.5 | 1.5 | 0 | 0.75 |
| EXOTHERMIA | 2.75 | 1.625 | 2 | 0 | 0.625 |
| EFFERVESCENCE | NO | YESì | YES | NO | NO |

### Example 5

### Stability Test for Combinations SAMe+Buffering Agents.

To test the good stability of the association SAMe and L-Arginine, and compare it with the stability of the other buffering agents, the mixtures used in Example 3 were placed in a climatic chamber within heat-sealed aluminum bags, at climatic conditions of 50° C. for 5 days, to test the stability of the mixtures.

The control parameters were:
Appearance
% H₂O determined by KF (Karl Fischer) method
Active assay
Degradation substances

Table 12 shows that not all buffering agents ensure proper stability of the active ingredient.

TABLE 12

| | BATCH: 13919-4 PR.1 | | BATCH: 13919-4 PR.2 | | BATCH: 13919-4 PR.3 | | BATCH: 13919-4 PR.4 | | BATCH: 13919-4 PR.5 | |
|---|---|---|---|---|---|---|---|---|---|---|
| | TIME | STEP 1: | TIME | STEP 1: | TIME | STEP 1: | TIME | STEP 1: | TIME | STEP 1: |
| ANALYSIS | ZERO | 5 | ZERO | 5 days | ZERO | 5 days | ZERO | 5 days | ZERO | 5 days |
| APPEARANCE | White | Unchanged | White | Unchanged | White | Unchanged | White | Unchanged | White | Unchanged |
| | powder | | powder | | powder | | powder | | powder | |
| % H₂O K.F | 1.581% | 1.681% | N.D * | N.D* | N.D* | N.D* | 3.934% | 4.106% | 2.030% | N.D |
| ASSAY | 30.30% | 29.29% | 36.56% | 30.53% | 19.70% | 14.07% | 23.79% | 21.53% | 49.84% | 48.39% |
| | | (−3%) | | (−16%) | | (−28.6%) | | (−9%) | | (−2.9%) |
| DEGRAD. | 0.61% A | 3.15% A | 1.05% A | 4.85% A | 1.25% A | 4.94% A | 2.68% A | 7.45% A | 1.03% A | 3.27% A |
| SUBST. | (0.05 P/P) | (0.25 P/P) | (0.11 P/P) | (0.43 P/P) | (0.07 P/P) | (0.19 P/P) | (0.21 P/P) | (0.45 P/P) | (0.14 P/P) | (0.42 P/P) |
| LOD 45° C. × | — | — | 1.23% | 1.00% | 0.68% | 0.45% | — | — | 0.75% | 0.80% |
| 20′ | | | | | | | | | | |

| | | | | |
|---|---|---|---|---|
| STABILITY: | STABILITY: | STABILITY: | STABILITY: | STABILITY: |
| NON- | CRITICAL | CRITICAL | NON- | NON- |
| CRITICAL | | | CRITICAL | CRITICAL |

### Example 6

### Stability Test on SAMe+Arginine Swallowable Tablet

To further confirm the stabilizing effect of Arginine on active ingredients with acid pKa, a stability test was performed on a swallowable tablet of SAMe and Arginine according to Example no. 8.

The tablet was obtained using standard equipment known to the person skilled in the art following a process of weighing, mixing, and pressing the mixture.

The tablets thus obtained in Example no. 8 were placed in a climatic chamber within heat-sealed aluminum bags, under climatic conditions of 50° C. for 5 days to test the tablets stability by monitoring the parameters at T0, T2, T5

The control parameters were:
Appearance
% H₂O determined by KF (Karl Fischer) method
Active assay
Degradation substances

Table 13 demonstrates that tablets thus obtained show an excellent stability profile thanks to the presence of arginine as a stabilizer.

TABLE 13

| | BATCH: 3520-1B | | |
|---|---|---|---|
| ANALYSIS | TIME ZERO | STEP 1: 5 | STEP 1: 5 |
| APPEARANCE | White Tablet | Unchanged | Unchanged |
| % H₂O K.F | 0.819% | 0.799% | 0.837% |
| ASSAY | 205.53 mg/cpr | 197.13 mg/cpr | 197.40 mg/cpr |
| | | (−4.1%) | (−4%) |
| DEGRAD. SUBST. | 1.08% A | 2.92% A | 6.04% A |

STABILITY: NON-CRITICAL

1. Use of arginine and/or a pharmaceutically acceptable salt thereof as a stabilizer of an acid active ingredient having a pKa of between 1 and 5.

2. Use of arginine and/or a pharmaceutically acceptable salt thereof according to claim 1, characterized in that said arginine is selected from L-arginine, D-arginine, and DL-arginine.

3. Use of arginine and/or a pharmaceutically acceptable salt thereof of claim 1, characterized in that said active ingredient is selected from S-Adenosyl-I-methionine or a salt thereof, ascorbic acid, acid amino acids, such as glutamic acid, N-acetyl-carnitine, L-acetyl-cysteine, acetyl-salicylic acid, salicylic acid, S-acetyl-l-glutathione, aspartic acid, lipoic acid, hydroxy-methyl-butyrate, carnitine, and acid botanical extracts, such as extracts of acerola and dog rose.

4. Use of arginine and/or a pharmaceutically acceptable salt thereof according to claim 3, characterized in that said active ingredient is S-Adenosyl-I-methionine.

5. Edible formulation comprising an acid active ingredient having a pKa of between 1 and 5, arginine and/or a pharmaceutically acceptable salt thereof, and at least one physiologically acceptable excipient.

6. Edible formulation according to claim 5, characterized in that said active ingredient has a pKa of between 1 and 4.5.

7. Edible formulation according to claim 6, characterized in that said arginine is selected from L-arginine, D-arginine, and DL-arginine.

8. Edible formulation according to claim 6, characterized in that said active ingredient is selected from S-Adenosyl-l-methionine or a salt thereof, ascorbic acid, acid amino acids, such as glutamic acid, N-acetyl-carnitine, L-acetyl-cysteine, acetyl-salicylic acid, salicylic acid, S-acetyl-l-glutathione, aspartic acid, lipoic acid, hydroxy-methyl-butyrate, carnitine, or acid botanical extracts, such as extracts of acerola and dog rose.

9. Edible formulation according to claim 8, characterized in that said active ingredient is S-Adenosyl-l-methionine.

10. Edible formulation according to claim 5, characterized in that said at least one physiologically acceptable excipient is selected from diluents, lubricants, aggregating agents, disintegrating agents, film forming agents, coloring agents, sweeteners or flavoring agents and/or antioxidants-antimicrobials.

11. Edible formulation according to claim 10, characterized in that said at least one physiologically acceptable excipient is present in an amount of 5-80% by weight, with respect to the total weight of the formulation.

12. Edible formulation according to claim 5, characterized by being in the form of orosoluble formulations, preferably selected from orodispersible tablets, sublingual tablets, powders or granules for sachets or stick-packs; or in the form of swallowing formulations, preferably selected from chewable tablets, drinkable solutions, or syrups.

13. Edible formulation according to claim 5, characterized in that said active ingredient is present in an amount ranging from 5 to 70% by weight, with respect to the total weight of the formulation.

14. Edible formulation according to claim 5, characterized in that arginine or a pharmaceutical salt thereof is present in an amount ranging from 5% to 70% by weight, with respect to the total weight of the formulation.

15. Edible formulation according to claim 5, characterized in that it contains at least one further active ingredient.

16. Edible formulation according to claim 15, characterized in that said further active ingredient has a dietetic, alimentary, nutraceutical and/or phytotherapeutic activity and is selected from vitamins, such as vitamins A, D, E, K, vitamins of group B, pantothenic acid, minerals, such as magnesium, calcium, phosphorus, iron, zinc, copper, manganese, fluorine, selenium, chromium, molybdenum, iodine, boron, potassium, chlorine, sodium, silicon salts, phytotherapeutic agents, such as botanical extracts of Curcuma longa, Boswellia serrata, Silybum marianum, Ananas comosus, Griffonia simplicifolia, Camelia sinensis, Olea europea and other substances with a nutritional and/or physiological effect, such as essential amino acids, branched amino acids, hydroxy-isocaproic acid (HICA), hyaluronic acid, conjugated linoleic acid (CLA), nervonic acid, alpha-ketoisocaproate (KIC), arabinogalactan, arabinoxylan, arginine alpha-ketoglutarate (AAKG), astaxanthin, beta-alanine, betaine, beta-glucans, butyrate, caffeine, carnosine, chitosan, citicoline, chlorophyll, coenzyme Q10, ubiquinol, choline, collagen, colostrum, chondroitin sulfate, creatine, dimethylglycine, enzymes such as alpha-galactosidase, bromeline, enzymes from fermented maltodextrins, lactase, beta-galactosidase, papain or superoxide dismutase, epigallocathechingallate, phytosterols, flavonoids such as quercetin, quercitrin, rutin, spireoside, hesperidin, hesperitin or diosmin, phospholipids such as phosphatidylcholine, phosphatidylserine or phosphoserine, GABA, gamma oryzanol, glycero-phosphoryl-ethanolamine, glycocyamine, glucomannan, glucosamine, glucuronolactone, glutathione, guar gum, hydroxytyrosol/olive polyphenols, inositol, isoflavones, lactoferrin, lactulose, lycopene, lutein, melatonin, methylsulfonylmethane (MSM), monacolin K from fermented red rice (Monacus purpureus), N-acetyl-D-glucosamine, NADH, naringin, norvaline, nucleotides, fish oil (DHA-EPA), homotaurine, ornithine alpha-ketoglutarate (OKG), palmitoylethanolamide (PEA), PABA, pectin, pycnogenol, policosanol, polydatin, resveratrol, ribose, spermidine, squalene, taurine, theanine, zeaxanthin, or mesozeaxanthin.
